(19) United States
(12) Patent Application Publication
(10) Pub. No.: US 2026/0240903 A1
(43) Pub. Date: Aug. 20, 2026

(54) USE OF TRIVALENT CHROMIUM IONS AND/OR METAL CHROMIUM IN PREPARATION OF DRUG FOR IMMUNOTHERAPY OF TUMORS

(71) Applicant: SHENZHEN LUOHU PEOPLE'S HOSPITAL, Shenzhen, Guangdong (CN)

(72) Inventors: Quan LIU, Shenzhen, Guangdong (CN); Dixian LUO, Shenzhen, Guangdong (CN); Jing WANG, Shenzhen, Guangdong (CN); Yu LIU, Shenzhen, Guangdong (CN)

(73) Assignee: SHENZHEN LUOHU PEOPLE'S HOSPITAL, Shenzhen, Guangdong (CN)

(21) Appl. No.: 19/159,835

(22) PCT Filed: Apr. 13, 2023
(86) PCT No.: PCT/CN2023/087964

§ 371 Date: Aug. 26, 2025

(30) Foreign Application Priority Data
Feb. 27, 2023 (CN) 202310167455.1

(51) Int. Cl. A61K 33/24; A61K 31/44; A61K 40/15; A61K 40/31; A61P 35/00

(52) U.S. Cl. CPC A61K 33/24; A61K 31/44; A61K 40/15; A61K 40/31; A61P 35/00

(57) ABSTRACT

Use of trivalent chromium ions and/or metal chromium in the preparation of a drug for the immunotherapy of tumors. The chromium metal ions have a metal immune effect, can improve the migration and infiltration of immune cells in tumors, can solve the problem whereby the immune cells are difficult to migrate and infiltrate in tumor tissues, can improve the immune microenvironment in tumors and increase the number of immune cells, such as dendritic cells DC, M1 type macrophages, CAR-T cells, iPS-NK cells, CD4+T cells and CD8+T cells, has the potential of activating the immunity of the body, and can effectively synergize and enhance the efficacy of the immunotherapies of PD-1/L1, such as ICB therapy, antibody therapy, cell therapy and a tumor vaccine.

### TECHNICAL FIELD

The present application belongs to the field of biomedicine and relates to a use of trivalent chromium ions and/or metal chromium in preparation of a drug for an immunotherapy of tumors.

### BACKGROUND

Immunotherapy is an important means for cancer treatment with immune checkpoint blockage (ICB) (for example, PD-1 and PD-L1 antibodies) as the main means. An immunotherapy effect of solid tumors is mainly limited by two factors: (1) immunosuppression with a PD-1/L1 signaling pathway as the main signaling pathway; and (2) the small number of tumor-infiltrating lymphocytes (TILs) or anti-tumor immune cells. The PD-1/L1 antibody has achieved the rebalancing of the intratumoral anti-tumor immunity. However, a therapeutic effect is still severely affected due to the excessively small number of TILs or immune cells. Therefore, how to improve the intratumoral immune environment and increase the number of anti-tumor immune cells is the main scientific problem in improving the efficacy of an immunotherapy of cancers.

Metal immunity is a new direction for an immunotherapy of tumors and aims to improve the body's immune response and an intratumoral immune microenvironment through metal ions. Metal ions have a single component, strong quality control and great potential for transformation. A role of the metal ions in immunotherapy has been explored. For example, Mn²⁺ activates the STING pathway of immune cells, and Mg²⁺ improves the killing activity of CD8⁺T cells. Therefore, other single elements such as selenium, phosphorus, boron, selenium and manganese may have the potential to activate the body's immunity.

Based on these research backgrounds, finding out metal ions that can improve the intratumoral immune environment, increase the number of immune cells or TILs or engineered immune cells and have the potential to activate the body's immunity has important clinical significance. Immunotherapy products and treatment means developed with metal ions with above potential have significant clinical significance.

### SUMMARY

The present application provides a use of trivalent chromium ions and/or metal chromium in preparation of a drug for an immunotherapy of tumors.

In a first aspect, the present application provides a use of trivalent chromium ions and/or metal chromium in preparation of a drug for an immunotherapy of tumors.

Chromium (Cr) is a micronutrient that can improve insulin resistance, type 2 diabetes and adaptive immune diseases. Cr³⁺ promotes the binding of insulin to macrophage insulin receptors (IRs) and enhances the activity of IR tyrosine kinases by six times and glucose metabolism. Nano-chromium is non-toxic to the body and mainly degraded into Cr³⁺ in vivo, while toxic Cr⁶⁺ is unstable and rapidly converted into Cr³⁺.

The present application creatively discovers that with an outstanding metal immune effect compared with other metal ions, chromium metal ions can significantly improve the intratumoral migration and infiltration of immune cells (for example, lymphocytes), can solve the core bottleneck problem of immunotherapy-difficult migration and infiltration of immune cells into intratumoral tissues, can improve an intratumoral immunosuppressive microenvironment, increase the number of immune cells such as TILs, have the potential to activate the body's immunity and have important pioneering and innovative significance for the field of immunotherapies of tumors. In addition, materials such as nano-chromium prepared from metal chromium can be degraded into trivalent chromium ions in vivo. Therefore, metal chromium can also be used for the preparation of the drug for the immunotherapy.

In a second aspect, the present application provides a combined pharmaceutical composition for an immunotherapy of tumors. The combined pharmaceutical composition includes a pharmaceutical component prepared from trivalent chromium ions and/or metal chromium, and further includes any one or a combination of at least two of an ICB drug, a cell therapy drug or a tumor vaccine.

Preferably, the pharmaceutical component prepared from metal chromium includes nano-chromium prepared through a preparation method including the following steps:
mixing metal chromium with a dispersing agent to obtain a dispersion, centrifuging the dispersion, and collecting a precipitate to obtain nano-chromium.

Preferably, the dispersing agent includes isopropanol.

Preferably, the mixing is performed under an ultrasound.

Preferably, the ultrasound is performed at power of 300-1000 W (for example, 300 W, 320 W, 340 W, 360 W, 380 W, 400 W, 420 W, 450 W, 500 W, 550 W, 600 W, 650 W, 700 W, 750 W, 800 W, 850 W, 900 W, 950 W or 1000 W, etc.) for a period of 8-12 h (for example, 8 h, 8.5 h, 9 h, 9.5 h, 10 h, 10.5 h, 11 h, 11.5 h or 12 h, etc.).

Preferably, the centrifuging is performed at a speed of 1000-10000 g (for example, 1000 g, 2000 g, 3000 g, 4000 g, 5000 g, 6000 g, 7000 g, 8000 g, 9000 g or 10000 g, etc.) for a period of 30-100 min (for example, 30 min, 40 min, 50 min, 60 min, 70 min, 80 min, 90 min or 100 min, etc.).

Preferably, the ICB drug includes a PD-1/PD-L1 inhibitor and/or a PD-1/PD-L1 antibody.

Preferably, the cell therapy drug includes any one or a combination of at least two of a CAR-T, a CAR-NK, a virus or a microorganism.

Preferably, the CAR-NK includes a CAR-NK derived from an organism and/or a CAR-NK derived from artificially synthesized iPS.

Preferably, the combined pharmaceutical composition includes a nano-chromium-inhibitor complex prepared through a preparation method including the following steps:
mixing nano-chromium, a PD-1/PD-L1 inhibitor, a solvent, dopamine and sodium hydroxide, performing a reaction to obtain a mixture, centrifuging the mixture, and collecting a precipitate to obtain the nano-chromium-inhibitor complex.

Preferably, the solvent includes ethanol.

Preferably, the reaction is performed under a dark condition.

Preferably, the reaction is performed at a temperature of 15-40° C. (for example, 15° C., 20° C., 25° C., 30° C., 35° C. or 40° C., etc.) for a period of 3-6 h (for example, 3 h, 3.5 h, 4 h, 4.5 h, 5 h, 5.5 h or 6 h, etc.).

Preferably, the centrifuging is performed at a rotation speed of 8000-16000 rpm (for example, 8000 rpm, 9000 rpm, 10000 rpm, 11000 rpm, 12000 rpm, 13000 rpm, 14000 rpm, 15000 rpm or 16000 rpm, etc.) for a period of 1-10 min (for example, 1 min, 2 min, 3 min, 4 min, 5 min, 6 min, 7 min, 8 min, 9 min or 10 min, etc.).

Preferably, the tumors include melanoma (including melanoma lung metastasis) or liver cancer.

Preferably, the combined pharmaceutical composition is a single compound preparation or a combination of at least two individual preparations.

Preferably, when the combined pharmaceutical composition is a combination of at least two individual preparations, the administration of the at least two individual preparations may be simultaneous administration, cross administration or sequential administration.

Preferably, the preparation is any one of the pharmaceutically acceptable dosage forms such as a tablet, a powder, a suspension, a granule, a capsule, a solution, an enema, an emulsion or a gel.

Preferably, the combined pharmaceutical composition further includes a pharmaceutically acceptable adjuvant.

Preferably, the pharmaceutically acceptable adjuvant includes any one or a combination of at least two of a carrier, a diluent, an excipient, a filler, an adhesive, a wetting agent, a disintegrating agent, an emulsifier, a cosolvent, a solubilizer, an osmotic pressure regulator, a surfactant, a coating material, a colorant, a pH adjusting agent, an anti-oxidant, a bacteriostatic agent or a buffer.

Preferably, the carrier includes any one or a combination of at least two of a hydrogel, a liposome or a nanocarrier.

In a third aspect, the present application provides a drug for an immunotherapy of tumors combined with photothermal therapy. The drug for the immunotherapy of the tumors combined with the photothermal therapy includes any one or a combination of at least two of trivalent chromium ions, a pharmaceutical component prepared from metal chromium or the combined pharmaceutical composition described in the second aspect.

Preferably, the pharmaceutical component prepared from metal chromium includes nano-chromium. A method for preparing the pharmaceutical component is the same as that described in the second aspect and is not repeated.

Preferably, a light source used in the photothermal therapy includes near-infrared light.

Preferably, the near-infrared light has a wavelength of 780-1100 nm.

In a fourth aspect, the present application provides a use of trivalent chromium ions and/or metal chromium in preparation of a drug for promoting intratumoral migration and/or infiltration of immune cells.

Preferably, the drug includes any one or a combination of at least two of a drug for promoting the intratumoral migration and/or infiltration of macrophages, a drug for promoting the intratumoral migration and/or infiltration of lymphocytes, a drug for promoting the intratumoral migration and/or infiltration of dendritic cells or a drug for promoting the intratumoral migration and/or infiltration of immune cells obtained through engineering with an immunosynthetic method.

In a fifth aspect, the present application provides a method for promoting intratumoral migration and/or infiltration of immune cells. The method includes: co-incubating immune cells with a pharmaceutical component prepared from trivalent chromium ions and/or metal chromium to promote migration and/or infiltration of the immune cells.

Preferably, the immune cells include, but are not limited to, any one or a combination of at least two of macrophages, lymphocytes or dendritic cells, NK cells, immune cells prepared through immunosynthetic engineering or immune cells derived from iPS.

Preferably, the immune cells include, but are not limited to, any one or a combination of at least two of human or mouse bone marrow-derived dendritic cells, human or mouse monocytic leukemia macrophage cells, human or mouse spleen lymphocytes, human or mouse CAR-T cells, human or mouse CAR-NK or immune cells derived from human or mouse iPS.

Any numerical range described in the present application includes not only the above-listed point values but also any point values within the numerical range which are not listed. Due to the limitation of space and the consideration of simplicity, specific point values included in the range are not exhaustively listed in the present application.

Compared with the existing art, the present application has the beneficial effects below.
(1) The present application creatively discovers that with the outstanding metal immune effect compared with other metal ions, chromium metal ions can significantly improve the intratumoral migration and infiltration of immune cells (for example, lymphocytes), can solve the core bottleneck problem of the immunotherapy-difficult migration and infiltration of immune cells into intratumoral tissues, can improve the intratumoral immunosuppressive microenvironment, increase the number of immune cells such as TILs, have the potential to activate the body's immunity and have the important pioneering and innovative significance for the field of immunotherapies of tumors.
(2) Materials such as nano-chromium prepared from metal chromium can be degraded into trivalent chromium ions in vivo. Therefore, metal chromium can also be used for the preparation of the drug for the immunotherapy.
(3) The present application also creatively discovers that materials such as chromium metal ions or nano-chromium and the ICB drug (the PD-1/PD-L1 inhibitor) play a synergistic role in treating tumors. Moreover, combining with immunotherapies such as the PD-1 antibody to treat tumors can improve an intratumoral immune environment, significantly enhance the infiltration of T lymphocytes and M1 tumor cells into tumor tissues and significantly promote the effective activation of an anti-tumor immune response, finally achieving effective inhibition of tumor growth and metastasis and achieving an object of treating cancers.
(4) The present application also creatively discovers that combining the materials such as chromium metal ions or nano-chromium with the photothermal therapy can significantly improve a therapeutic effect of tumors (for example, melanoma and melanoma lung metastasis).
(5) In addition, combining with the photothermal therapy on the basis of combining the materials such as chromium metal ions or nano-chromium with the ICB drug (the PD-1/PD-L1 inhibitor) can further improve the therapeutic effect of the tumors (for example, melanoma and melanoma lung metastasis).

### BRIEF DESCRIPTION OF DRAWINGS

FIG. 1 is a diagram illustrating results of evaluating the migration ability of different concentrations of metal ions to macrophages (RAW264.7) in a Transwell research.

FIG. 2 is a diagram illustrating results obtained after toxic and side effects of different metal ions on tumor cells are evaluated through CCK8, where A is a diagram illustrating results of Cr³⁺, Fe³⁺, Fe²⁺, K⁺, Ca²⁺ and Mg²⁺, and B is a diagram illustrating results of Al3+, Mn2+, Co2+, Cu²⁺ and Zn²⁺.

FIG. 3 is a diagram illustrating results obtained after Cr³⁺ inhibits the growth of subcutaneous melanoma in combination with a PD-1/PD-L1 inhibitor BMS-202.

FIG. 4 is an image of tumors of mice in different treatment groups.

FIG. 5 is a diagram illustrating results obtained after Cr³⁺ with different concentrations detects the secretion of MIP-1a in RAW264.7 cells.

FIG. 6 is an image illustrating results obtained after immunoblotting is performed with anti-PI3K, AKT, phosphorylated AKT and an mTOR antibody.

FIG. 7 is an XPS spectrum detection diagram illustrating that nano-chromium is mainly degraded into trivalent chromium ions in vitro.

FIG. 8 is a Raman spectrum illustrating that nano-chromium is mainly degraded into trivalent chromium ions in the presence or absence of an acid.

FIG. 9 is a schematic diagram illustrating that the promotion of the migration of immune cells by Cr nanoparticles is measured through a Transwell method.

FIG. 10 is a diagram illustrating results obtained after abilities of Cr nanoparticles to promote BM-DCs and macrophages to migrate are evaluated.

FIG. 11 is a histogram illustrating that Cr nanoparticles promote the migration of macrophages.

FIG. 12 is a histogram illustrating that Cr nanoparticles promote the migration of BM-DCs.

FIG. 13 is a histogram illustrating that Cr nanoparticles promote the migration of iPS-NK cells.

FIG. 14 is a diagram illustrating results of lung metastases of mice in different treatment groups.

FIG. 15 is a diagram illustrating results of lung pathological sections.

FIG. 16 is a diagram illustrating statistical results of lung metastatic tumor foci.

FIG. 17 is a statistical diagram illustrating the survival of mice in metastasis models of different treatment groups.

FIG. 18 is a diagram illustrating results obtained after immunohistochemical staining is performed on iNOS (M1 macrophages) and CD8 (CD8⁺T cells) in lung tissues.

### DETAILED DESCRIPTION

Technical solutions of the present application are further described below through specific examples. Those skilled in the art are to understand that the examples described herein are used for a better understanding of the present application and are not to be construed as specific limitations to the present application.

In the following examples, unless otherwise specified, all the reagents and consumables are purchased from conventional reagent manufacturers in the art; unless otherwise specified, the experimental methods and technical means used are conventional methods and means in the art.

### EXAMPLE 1

This example provides the establishment and validation of a platform for screening metal ions that promotes the migration of immune cells. A specific method is described below.
1. Preparation of metal ions: metal compounds such as zinc chloride, potassium chloride, magnesium chloride, manganese chloride, anhydrous calcium chloride, aluminium sulfate (octadecahydrate), anhydrous copper(II) chloride, anhydrous iron(III) chloride, iron(II) chloride, chromium(III) chloride hexahydrate and anhydrous cobalt(II) chloride were prepared into solutions ranging from 0 μM to 2000 μM using normal saline, respectively.
2. Preparation of BM-DCs (bone marrow-derived dendritic cells), RAW264.7 (mouse monocytic leukemia macrophage cells) and mouse spleen lymphocytes
\textcircled{1} Acquisition of mouse BM-DCs: bone marrow was taken from hind limbs of C25BL/6 mice, and treated with a red blood cell lysis buffer to remove red blood cells; bone marrow cells (4×10⁶ cells) were cultured in a 10 cm culture dish, and 10% FBS, mercaptoethanol (2-ME, 0.8 ng/ml, Sigma) and mouse granulocyte-macrophage colony-stimulating factor (GM-CSF, 20 ng/mL, PeproTech) were added to a DMEM medium and cultured for 7 days; expression levels of CD11c, CD80 and CD86 in cells were detected using antibodies and a flow cytometer.
\textcircled{2} Mouse macrophages RAW264.7: derived from a cell bank of the Chinese Academy of Sciences and cultured.
\textcircled{3} Mouse spleen lymphocytes: spleens of C57BL/6 mice were acquired and filtered by a screen into single cells, treated with a red blood cell lysis buffer to remove red blood cells, and centrifuged with lymphocyte isolation solution to isolate the cells for in vitro culture.
3. Transwell detection of the promotion of the migration of immune cells by metal ions

Liver cancer cells were placed in lower chambers (12 wells) of a Transwell plate, and the above metal ion solutions with different concentrations (0 μM, 500 μM, 1000 μM and 2000 μM) were added to the lower chambers, respectively. After 24 h, the BM-DCs, the RAW264.7 cells and the spleen lymphocytes were placed in upper chambers (2×10⁴ cells/well) of the Transwell plate, respectively. The cells were co-cultured for a certain period of time, and nylon membranes of the chambers were taken and stained to observe the migration of the cells.

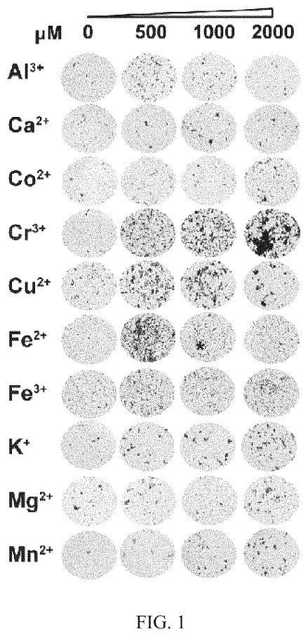

FIG. 1 is a diagram illustrating results obtained after abilities of metal ions with different concentrations to promote macrophages (RAW264.7) to migrate in vitro are shown in a Transwell research. As can be seen from the figure, Cr³⁺, Cu²⁺ and Fe²⁺ have an ability to promote the migration of macrophages. Cr³⁺ between 0 μM and 2000 μM has a significant migration promoting effect.

### EXAMPLE 2

This example evaluates toxic and side effects of different metal ions on tumor cells.

Toxicities of metal ions with different concentrations on RAW264.7 cells were quantified through a CCK-8 assay. Specific steps are as follows: RAW264.7 cells (2×10⁴ cells/well, n=4) were inoculated in a 96-well plate, stayed for a whole night and cultured by media containing metal ions with different concentrations (0 μg/mL, 25 μg/mL, 50 μg/mL, 100 μg/mL and 200 μg/mL) for 24 h, respectively, and cell viability was measured through CCK-8.

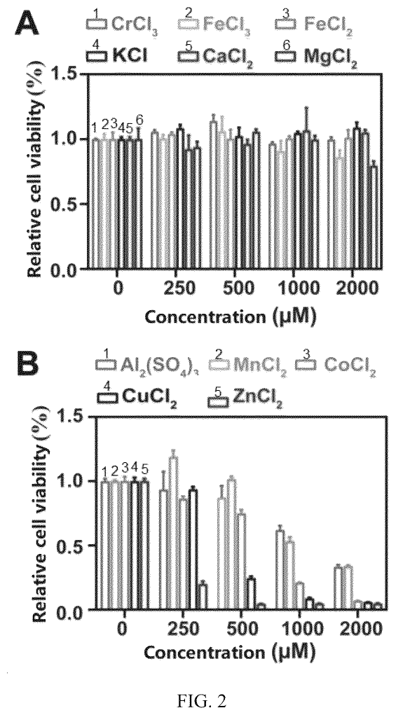

The results of the toxic and side effects of the different metal ions on the RAW264.7 cells are shown in FIG. 2. As can be seen from the figure, Cr³⁺ has good biocompatibility (low biological toxicity) and immunostimulatory effects, Cu²⁺ has relatively high cytotoxicity, and Fe²⁺ has poor stability in vitro and in vivo and is easily oxidized to Fe³⁺.

### EXAMPLE 3

This example evaluates an inhibitory effect of Cr³⁺ on the growth of subcutaneous melanoma in combination with a PD-1/PD-L1 inhibitor BMS-202.

A batch of SPF C57BL/6 mice (6 weeks old) were purchased from a company. B16F10 cells were selected as subcutaneous tumor model cells. 5×10⁵ B16F10 cells were inoculated on a right side of a dorsal flank of each mouse and cultured for 13 days. When tumor volumes reached 100-200 mm³, the modeling was successful. The obtained tumor-bearing mice can be used for a subsequent in vivo experiment. Animal experiments all comply with requirements for animal protection ethics and welfare.

The successfully constructed B16 tumor-bearing mice were divided into four groups: a CrCl₃ experimental group (amount of chromium ions: 100 μg/mL, 100 μL), a BMS experimental group (0.1 mg/mouse), a CrCl₃+BMS experimental group (100 μg/mL, 100 μL CrCl₃+0.1 mg BMS/mouse) and a control group (normal saline, 100 μL) (n=3 for each group). On the first day, CrCl₃, BMS and CrCl₃+BMS were injected into veins of mice in different groups, respectively. The treatment had an interval of 4 days and was performed twice in total. Tumor growth was monitored every two days by a digital caliper with a tumor volume calculation formula of ½×L×W². On the 12th day of treatment, the mice were euthanatized.

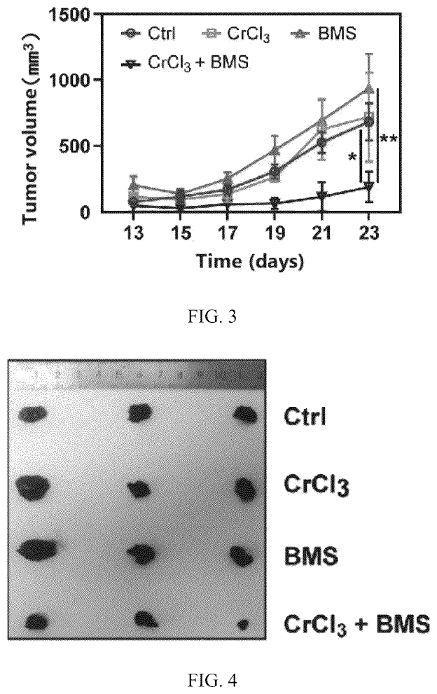

The results are shown in FIGS. 3 and 4 that are a statistical diagram of tumor sizes of mice in each group during the treatment (FIG. 3) and an anatomical chart of tumors of mice in each group (FIG. 4). As can be seen from the figures, using low-dose (0.1 mg/mouse) BMS-202 (BMS) or Cr³⁺ (100 μg/mL, 100 μL) alone has no significant limiting effect on the tumor growth, while using BMS in combination with Cr³⁺ can significantly inhibit the tumor growth of melanoma.

### EXAMPLE 4

This example evaluates an effect and mechanism that Cr³⁺ promotes the migration and tumor infiltration of immune cells.

Cr³⁺ with different concentrations (0 μg/mL, 50 μg/mL, 100 μg/mL and 200 μg/mL) was co-incubated with RAW264.7 cells (1×10⁶ cells/well) for 48 h. Cell supernatants and protein samples were collected, and various types of cells/chemokines including MIP-la were detected through ELISA. The expression of various proteins in a PI3K/Akt/mTOR pathway was detected through protein immunoblotting.

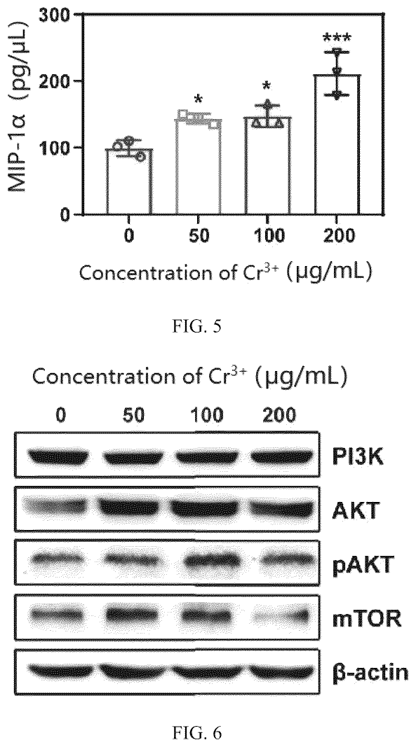

The results are shown in FIG. 5. RAW264.7 cells cultured in the Cr³⁺ with the different concentrations (0 μg/mL, 50 μg/mL, 100 μg/mL and 200 μg/mL) exhibit the concentration-dependent secretion of a macrophage inflammatory protein-1α (MIP-1α) that is increased as the concentration of ³⁺ is increased. The secretion of MIP-1α is significantly increased. MIP-1α is a typical cytokine that can promote the migration and tumor infiltration of macrophages, natural killer cells (NK), cytotoxic T cells and other immune cells.

In terms of mechanism, the detection results of the protein immunoblotting are shown in FIG. 6. The secretion of MIP-1α is regulated by the PI3K/AKT/mTOR pathway, and Cr³⁺ can participate in activating insulin receptors (IRs), upstream regulatory factors of the PI3K/AKT/mTOR pathway. The research results indicate that Cr³⁺ induces high expression of AKT and phosphorylated AKT (pAKT), thereby activating downstream mTOR. As can be seen from the figure, Cr³⁺ can activate the PI3K/AKT/mTOR pathway, thereby promoting the secretion of MIP-1α in the RAW264.7 cells.

### EXAMPLE 5

This example evaluates an ability of Cr³⁺ generated by nano-chromium to promote the migration of macrophages, DCs and iPS-CAR-NK cells.

A method for preparing Cr nanoparticles (Cr NPs) is as follows: two steps, probe sonication and water bath sonication, were performed on bulk chromium powder (Smart-Elements, Austria) in isopropanol. 20 mg chromium powder was mixed with 30 ml isopropanol. An on/off cycle of 2/2 seconds (power: 800 W, time: 9 h) was set for the probe sonication to avoid thermal oxidation during the sonication. A nano-chromium suspension was stored in ice water. Subsequently, the nano-chromium suspension was sonicated in a 360 W water bath for about 10 h and maintained at a temperature of 10° C. After these two sonication processes, the prepared dispersion was centrifuged at 1000 g for about 30 min to remove large nano-chromium. A supernatant containing the chromium nanoparticles was gently poured into a centrifuge tube, centrifuged at 8000 g for 30 min and dried in a vacuum dryer (room temperature that is around 25° C.), and the chromium nanoparticles were packaged by tin foil and stored at 4° C.

Degradation experiment: an experimental method is that after chromium powder or Cr nanoparticles were placed under an acidic condition (pH=5.5) or an acid-free condition and oxidized for two weeks, and oxidation products were detected using an XPS spectrum.

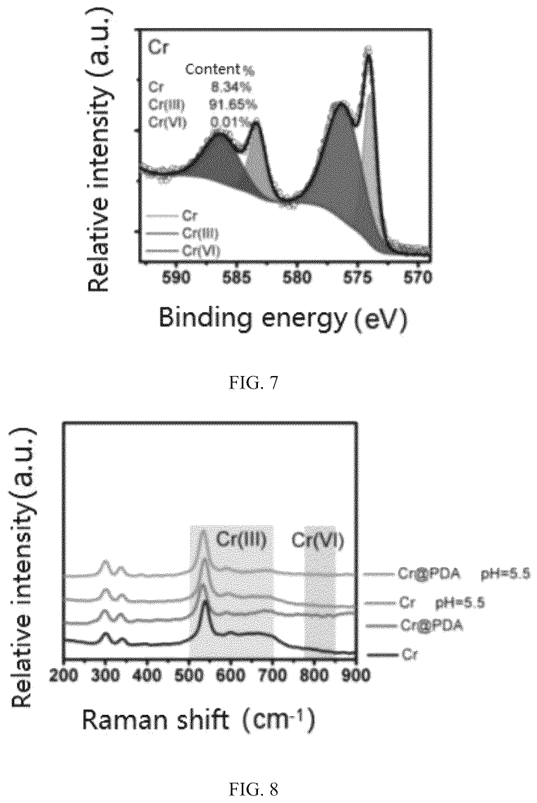

The results are shown in FIGS. 7 and 8 and indicate that the Cr nanoparticles are mainly degraded into non-toxic trivalent chromium ions (for an XPS spectrum detection diagram, see FIG. 7, and for a Raman spectrum, see FIG. 8).

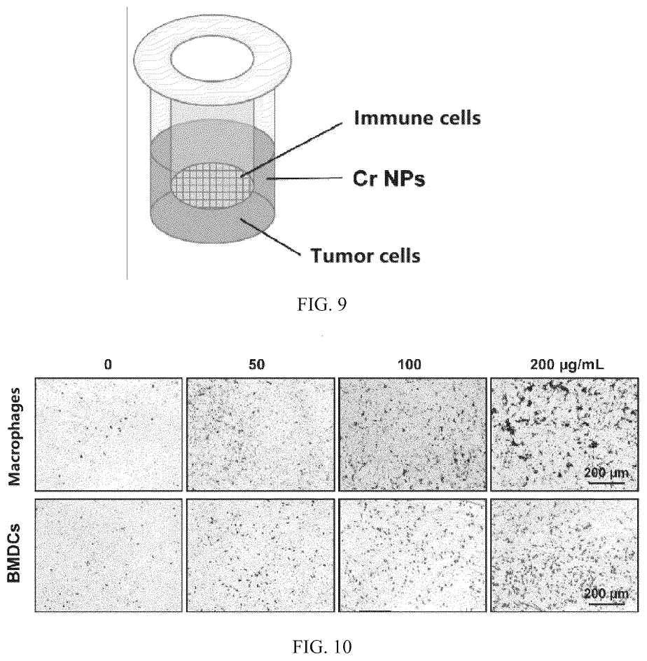

B16F10 cells were placed in lower chambers (12 wells) of a Transwell plate, and the above nano-chromium solutions with different concentrations were added to the lower chambers, respectively. After 24 h, RAW264.7 cells, mouse bone marrow-derived DCs (BM-DCs) and iPS-NK cells were placed in upper chambers (2×10⁴ cells/well) of the Transwell plate, respectively. The cells were co-cultured for 12-48 h (may be, for example, 12 h, 24 h, 30 h or 36 h), nylon membranes of the chambers were taken and stained, and migrating cells were labeled using crystal violet to observe the migration of the cells. The effects of the promotion of the migration of the immune cells by the Cr nanoparticles with the different concentrations were measured through a Transwell method (a schematic diagram is shown in FIG. 9).

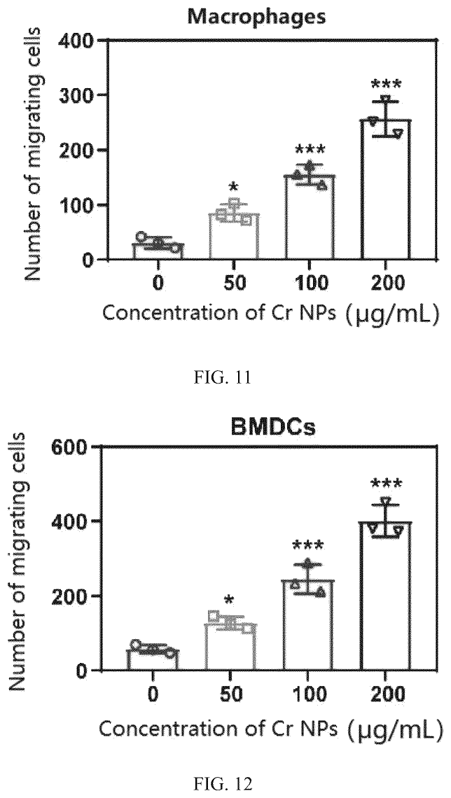

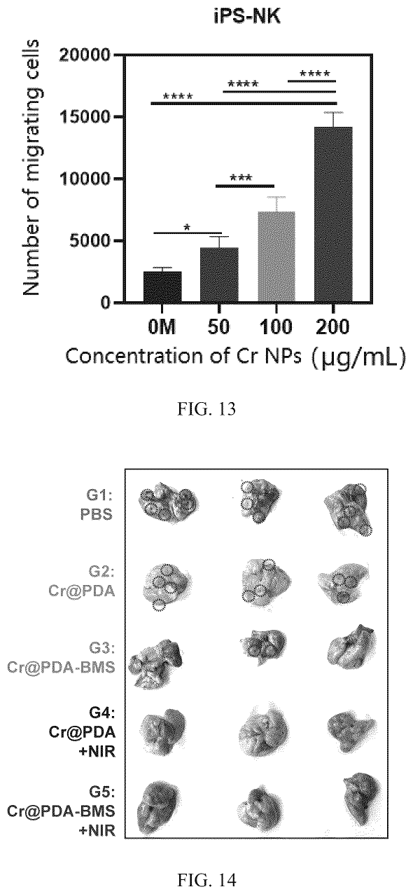

The migration results of the macrophages, the DCs and the iPS-NK cells are shown in FIGS. 10 to 13. The results indicate that compared with other groups, a migration ability of cells in a 200 μg/mL Cr nanoparticle treatment group is enhanced with significant statistical significance, suggesting that an ability of Cr³⁺ to promote the macrophages, the DCs and the iPS-NK cells to migrate is enhanced.

### EXAMPLE 6

This example evaluates an anti-tumor effect of a Cr nanosheet in combination with a PD-1/PD-L1 inhibitor BMS-202 on melanoma lung metastasis models.

A method for preparing Cr@PDA-BMS is as follows: 1 mg Cr NPs was suspended in 1 mL ethanol and uniformly dispersed through sonication, and 1.2 mg BMS-202 (BMS, Dalian MeilunBio®, China) was added and shaken to dissolve fully; subsequently, 20 μL aqueous solution of dopamine (100 mg/mL) was added, and 20 μL aqueous solution of sodium hydroxide (10 mg/mL) was added; the mixture was stirred in the dark at room temperature for 4 h and centrifuged at 12,000 rpm for 4 min; a precipitate was washed twice with water and stored at 4° C.

For a method for preparing Cr@PDA (Cr NPs coated with polydopamine), reference is made to the method for preparing Cr@PDA-BMS. The only difference is that BMS-202 was not added.

Establishment of animal models: a batch of healthy female C57BL/6 mice (5-6 weeks old, 16-20 g) under an SPF condition were purchased from a company to establish the melanoma lung metastasis models; 5×10⁵ B16F10 tumor cells were subcutaneously inoculated on a right side of each mouse; when tumor volumes approached 200 mm³, the mice were randomly divided into five groups and subjected to nanosystem-based therapies (n=6), respectively:
(1) control group (injection of normal saline into tail veins, 100 μL);
(2) injection of Cr@PDA into tail veins (amount of chromium element: 1 mg/kg, volume: 100 μL, dispersed in normal saline);
(3) injection of Cr@PDA-BMS into tail veins (amount of chromium element: 1 mg/kg, 100 μL; BMS: 1.2 mg/kg);
(4) injection of Cr@PDA into tail veins (amount of chromium element: 1 mg/kg, 100 μL) and an in vivo photothermal therapy (NIR, 808 nm, 1 W/cm², 8 min) after 24 h;
(5) injection of Cr@PDA-BMS into tail veins (amount of chromium element: 1 mg/kg, 100 μL) and an in vivo photothermal therapy (NIR, 808 nm, 1 W/cm², 8 min) after 24 h.

On the 7th day, the mice were subjected to the nanosystem-based therapies again. After two weeks, lungs of the mice were taken and examined for researches of tumor lung metastasis.

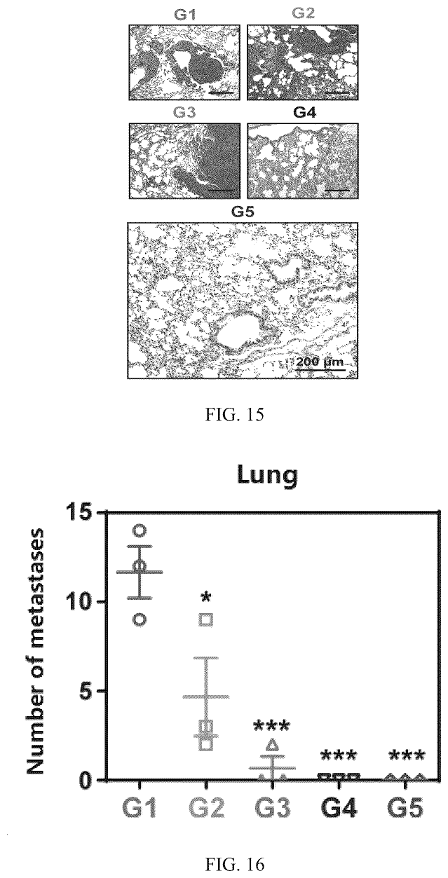

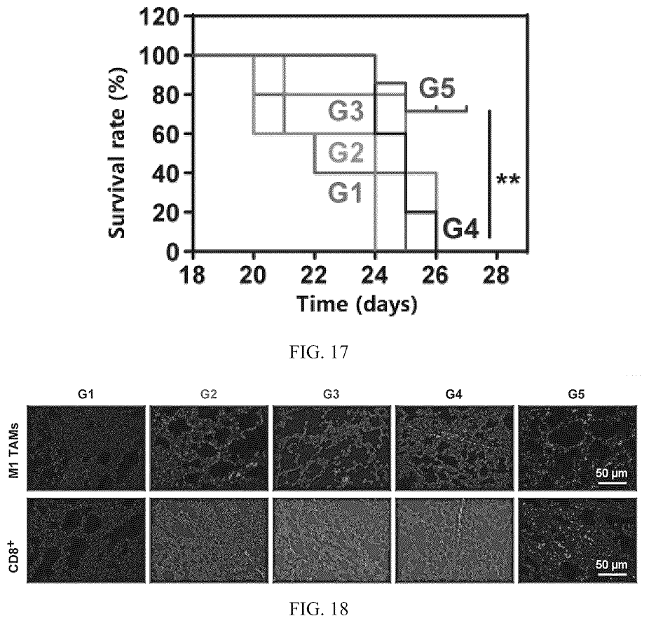

The results are shown in FIGS. 14 to 18, including lung metastases of mice in different treatment groups (FIG. 14), lung pathological sections for the observation of the formation of the metastases (FIG. 15), a diagram illustrating statistical results of lung metastatic tumor foci (FIG. 16), a statistical diagram illustrating the survival of mice in metastasis models of different treatment groups (FIG. 17) and a diagram illustrating results obtained after immunohistochemical staining is performed on iNOS (M1 macrophages) and CD8 (CD8⁺T cells) in lung tissues of mice in B16F10 lung metastasis models (FIG. 18).

As can be seen from the figures, compared with the control group, Cr@PDA in combination with the photothermal therapy and Cr@PDA-BMS in combination with the photothermal therapy have a significant inhibitory effect on melanoma lung metastasis and can improve the survival of the treated mice in the metastasis models. Moreover, compared with the control group, intratumoral infiltration rates of M1 TAMs (iNOS, red) and CD8⁺T cells in Cr@PDA and Cr@PDA-BMS groups are significantly improved. The results indicate that nano-chromium, a single element, can perform light-controlled killing on the tumor cells and activate immunity, and Cr³⁺, a degradation product of the nano-chromium, can significantly promote the intratumoral infiltration of the M1 macrophages and the CD8⁺T lymphocytes. Moreover, the applicant provides a concept of laser metal immunity of nano-chromium. That is, nano-chromium has both a laser photothermal property and a metal immune effect of promoting the intratumoral infiltration of immune cells. Through the above animal experiments, the applicant has verified that chromium has a significant laser metal immune effect and the effect is apparent in a combination use of an immunotherapy of tumors.

The applicant has stated that although the use of trivalent chromium ions and/or metal chromium in the preparation of the drug for the immunotherapy of the tumors in the present application are described through the preceding examples, the present application is not limited to the preceding examples, which means that the implementation of the present application does not necessarily depend on the preceding examples. It should be apparent to those skilled in the art that any improvements made to the present application, equivalent replacements of raw materials of the product of the present application, additions of adjuvant ingredients, selections of specific manners, etc., all fall within the protection scope and the disclosure scope of the present application.

Although the preferred examples of the present application have been described above in detail, the present application is not limited to details of the above-described examples, and various simple modifications can be made to the technical solutions of the present application without departing from the technical concept of the present application. These simple modifications are all within the protection scope of the present application.

In addition, it is to be noted that if not in collision, specific technical features described in the preceding specific examples may be combined in any suitable manner. To avoid unnecessary repetition, various possible combination manners are not further described in the present application.

1. (canceled)

2. A combined pharmaceutical composition for an immunotherapy of tumors, comprising a pharmaceutical component prepared from trivalent chromium ions and/or metal chromium, and further comprising any one or a combination of at least two of an ICB drug, a cell therapy drug or a tumor vaccine.

3. The combined pharmaceutical composition according to claim 2, wherein the pharmaceutical component prepared from metal chromium comprises nano-chromium prepared through a preparation method comprising the following steps:
mixing metal chromium with a dispersing agent to obtain a dispersion, centrifuging the dispersion, and collecting a precipitate to obtain nano-chromium;
wherein the dispersing agent comprises isopropanol;
wherein the mixing is performed under an ultrasound;
wherein the ultrasound is performed at power of 300-1000 W for a period of 8-24 h;
wherein the centrifugation is performed at a speed of 1000-10000 g for a period of 30-100 min.

4. The combined pharmaceutical composition according to claim 2, wherein the ICB drug comprises a PD-1/PD-L1 inhibitor and/or a PD-1/PD-L1 antibody;
wherein the cell therapy drug comprises any one or a combination of at least two of a CAR-T, a CAR-NK, a virus or a microorganism;
wherein the CAR-NK comprises a CAR-NK derived from an organism and/or a CAR-NK derived from artificially synthesized iPS.

5. The combined pharmaceutical composition according to claim 3, comprising a nano-chromium-inhibitor complex prepared through a preparation method comprising the following steps:
mixing nano-chromium, a PD-1/PD-L1 inhibitor, a solvent, dopamine and sodium hydroxide, performing a reaction to obtain a mixture, centrifuging the mixture, and collecting a precipitate to obtain the nano-chromium-inhibitor complex;
wherein the solvent comprises ethanol;
wherein the reaction is performed under a dark condition;
wherein the reaction is performed at a temperature of 15-40° C. for a period of 3-6 h;
wherein the centrifugation is performed at a rotation speed of 8000-16000 g for a period of 1-10 min.

6. The combined pharmaceutical composition according to claim 2, wherein the tumors comprise melanoma or liver cancer;
wherein the combined pharmaceutical composition is a single compound preparation or a combination of at least two individual preparations;
wherein the combined pharmaceutical composition further comprises a pharmaceutically acceptable adjuvant;
wherein the pharmaceutically acceptable adjuvant comprises any one or a combination of at least two of a carrier, a diluent, an excipient, a filler, an adhesive, a wetting agent, a disintegrating agent, an emulsifier, a cosolvent, a solubilizer, an osmotic pressure regulator, a surfactant, a coating material, a colorant, a pH adjusting agent, an anti-oxidant, a bacteriostatic agent or a buffer;
wherein the carrier comprises any one or a combination of at least two of a hydrogel, a liposome or a nanocarrier.

7. A drug for an immunotherapy of tumors combined with photothermal therapy, comprising any one or a combination of at least two of trivalent chromium ions, a pharmaceutical component prepared from metal chromium or the combined pharmaceutical composition according to claim 2.

8. The drug for an immunotherapy of tumors combined with the photothermal therapy according to claim 7, wherein a light source used in the photothermal therapy comprises near-infrared light;
wherein the near-infrared light has a wavelength of 780-1100 nm.

9. (canceled)

10. A method for promoting intratumoral migration and/or infiltration of immune cells, comprising: co-incubating immune cells with a pharmaceutical component prepared from trivalent chromium ions and/or metal chromium to promote migration and/or infiltration of the immune cells.

11. A method for an immunotherapy of tumors, comprising administering an effective amount of trivalent chromium ions and/or metal chromium to subject in need thereof.
